(19) United States
(12) Plant Patent Application Publication
(10) Pub. No.: US 2016/0014948 P1
(43) Pub. Date: Jan. 14, 2016

(54) Dianthus plant named 'KLEDG14174'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/545,533

(22) Filed: May 18, 2015

Related U.S. Application Data
(60) Provisional application No. 61/998,888, filed on Jul. 10, 2014.

(51) Int. Cl. A01H 5/00

(52) U.S. Cl. PLT/279

(57) ABSTRACT

A new Dianthus plant named ‘KLEDG14174’ particularly distinguished by white, double flowers with purple centers, medium growth, and an upright plant form, is disclosed.

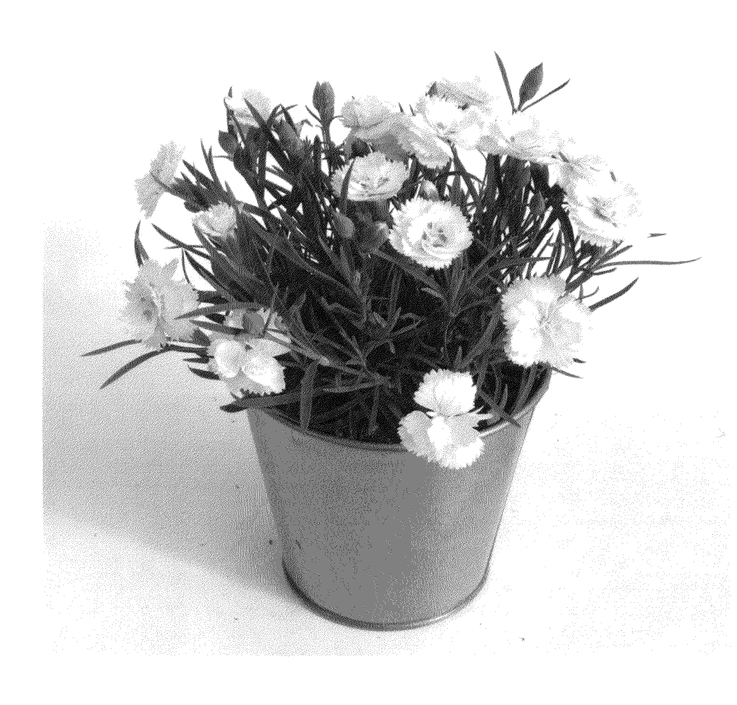

### CROSS REFERENCE TO RELATED APPLICATION

This application claims the benefit of U.S. Provisional Application No. 61/998,888 filed Jul. 10, 2014, the entire contents of which are incorporated herein by reference for all purposes.

Plant Breeder's Rights for ‘KLEDG14174’ were applied for in Europe on Jun. 3, 2014, Application No. 2014/1514. ‘KLEDG14174’ has not been made publicly available or sold more than one year prior to the filing date of this application.

### GENUS AND SPECIES

Dianthus sp.

### VARIETY DENOMINATION

‘KLEDG14174’

### BACKGROUND

The present invention comprises a new and distinct variety of Dianthus, botanically known as Dianthus sp. and hereinafter referred to by the variety name ‘KLEDG14174’. ‘KLEDG14174’ is a selection that resulted from a cross pollination of the proprietary female dianthus line ‘DSS 2007 0003’ (unpatented) and the male dianthus line ‘KLEDG10116’ (U.S. Plant Pat. No. 23,363).

In May 2010 the proprietary female dianthus line ‘DSS 2007 0003’ and the male dianthus line ‘KLEDG10116’ were cross pollinated in Stuttgart, Germany and seeds were obtained. The seeds were sown and plants were grown for evaluation. A plant line was selected in June 2011 in Stuttgart, Germany, and named ‘KLEDG14174’. In June 2011, ‘KLEDG14174’ was first vegetatively propagated by vegetative cuttings. ‘KLEDG14174’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. White, double flowers with purple centers;

2. Medium growth; and

3. An upright plant form.

### DESCRIPTION OF THE PHOTOGRAPH

These new Dianthus plant is illustrated by the accompanying photograph, which shows the overall plant habit of the variety ‘KLEDG14174’ grown in a pot. The photograph is of a 10 to 12 week old plant in Stuttgart, Germany grown under greenhouse conditions in March 2014. The colors shown are as true as can be reasonably obtained by conventional photographic procedures.

### DESCRIPTION OF THE VARIETY

The following detailed description sets forth the distinctive characteristics of ‘KLEDG14174’. The data which define these characteristics were collected from asexual reproductions carried out in Stuttgart, Germany. Data was collected on four month old plants grown in a glass greenhouse in Stuttgart, Germany in March, 2014. Color references are to The R.H.S. Colour Chart of The Royal Horticultural Society of London (R.H.S.), 5^{th} edition.
Classification:
   Family.—Caryophyllaceae.
   Species.—Dianthus sp.
   Common name.—Garden carnation.
   Parentage.—Female: The proprietary dianthus variety ‘DSS 2007 0003’ (unpatented) Male: The dianthus variety ‘KLEDG10116’ (U.S. Plant Pat. No. 23,363).
Propagation:
   Type.—Vegetative cuttings.
   Time to produce a rooted young plant.—About 20 days.
   Root description.—White, fine, well-branched.
   Rooting habit.—Lanceolate.
Plant description:
   Plant form.—Upright, bushy, round, potted and garden plant.
   Plant height.—12.0 cm.
   Plant diameter or spread.—20.0 cm.
Lateral branches:
   Length.—6.0 cm.
   Diameter.—1.5 cm.
   Internode length.—0.6 cm.
   Node length.—0.4 cm.
   Node diameter.—0.4 cm.
   Texture.—Smooth.
   Color.—RHS 137A.
Foliage description:
   Arrangement.—Opposite.
   Length.—6.0 cm.
   Width.—0.3 cm.
   Shape.—Lanceolate.
   Apex.—Acute.
   Base.—Obtuse.
   Margin.—Entire.
   Texture.—Upper and lower surfaces: Smooth.
   Color.—Immature and mature leaves: Upper surface: RHS 137C Lower surface: RHS 137D Mature leaves: Upper surfaces: RHS 137C Lower surface: RHS 137D.
   Venation pattern.—Pinnate.
   Venation.—RHS 138C.
Flower description:
   Flower type and habit.—Double, round.
   Fragrance.—Little.
   Natural flowering season.—Spring to autumn.
   Flower longevity.—8 to 10 days.
   Flower buds.—Length: 1.3 cm Diameter: 0.7 cm Shape: Ovate Color: RHS 137C.
   Corolla.—Quantity of petals/arrangement: More than 12 and having petaloids Corolla height: 2.0 cm Petaloids: Length: 0.6 cm Width: 0.5 cm Apex: Obtuse Base: Cuspidate Margin: Crenate Color: Upper surface: RHS NN155C and RHS 64B Lower surface: RHS NN155B Texture: Smooth Petal length: 3.0 cm Petal width: 1.4 cm Petal shape: V-shaped Petal apex: Obtuse Petal base: Cuspidate Petal margin: Crenate Petal texture: Upper and lower surfaces: Smooth Petal color: Immature: Upper surface: RHS NN155C and RHS 64B Lower surface: RHS N155A Mature: Upper surface: RHS NN155D and RHS 71B Lower surface: RHS NN155C Calyx: Quantity of sepals/arrangement: 5, single Calyx height: 1.5 cm Calyx diameter, at the apex: 0.6 cm Sepal texture: Upper and lower surfaces: Smooth Sepal color: Upper surface: RHS 138B Lower surface: RHS 138D.
Peduncles:
   Length.—8.0 cm.
   Diameter.—0.3 cm.
   Strength.—Moderate.
   Texture.—Smooth.
   Color.—RHS N138A.
Reproductive organs:
   Stamens.—Quantity: Less than 5 Filament length: 0.8 cm Filament color: RHS N155D Anther length: 0.2 cm Anther diameter: 0.08 cm Anther color: RHS N155B Pollen amount: Sparse.
   Pistils.—Quantity: 2 Pistil length: 1.6 cm Style length: 0.1 cm Style color: RHS NN155D Stigma shape: Filamentous Stigma color: RHS NN155C Ovary size: 0.4 cm Ovary shape: Ovate Ovary color: RHS 139D Fruits/seeds: None observed.
Disease and insect/pest resistance: No special observations were made

### COMPARISON WITH PARENTAL LINES AND KNOWN VARIETY

When ‘KLEDG14174’ is compared to the female and male parental lines, the following differences are noted in Table 1:

TABLE 1
Comparison with Parental Lines

| | Variety | Female parent | Male Parent |
|---|---|---|---|
| Trait | ‘KLEDG14174’ | ‘DSS 2007 0003’ | ‘KLEDG10116’ |
| Flower color | White with | White, with | Purple |
| | purple center | light pink | |
| Growth | Medium | Small, compact | Strong |
| Flower type | Double | Semi-double | Double |

When ‘KLEGD14174’ is compared to the commercial line Diantica ‘White’ (unpatented), the following differences are noted in Table 2:

TABLE 2
Comparison with Similar Variety

| | Variety | Commercial line |
|---|---|---|
| Trait | ‘KLEDG14174’ | Diantica ‘White’ |
| Plant form | Upright | Plain growing |
| Flower color | White with purple center | White with light pink center |

We claim:

1. A new and distinct variety of Dianthus plant named ‘KLEDG14174’ as illustrated and described herein.
